(19) United States
(12) Patent Application Publication
(10) Pub. No.: US 2004/0115223 A1
(43) Pub. Date: Jun. 17, 2004

(54) Prevention and treatment of allergies by helminthic regulation of IgE

(76) Inventor: David Follansbee, La Habra, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/719,532

(22) Filed: Nov. 21, 2003

Related U.S. Application Data
(63) Continuation of application No. PCT/US02/16517, filed on May 23, 2002.
(60) Provisional application No. 60/316,730, filed on Aug. 31, 2001.
(60) Provisional application No. 60/292,965, filed on May 23, 2001.

(51) Int. Cl. A61K 39/395; A61K 39/002

(52) U.S. Cl. 424/265.1; 424/152.1

(57) ABSTRACT

This invention relates to helminthic compositions in the prevention and treatment of allergies and asthma, and methods thereof. In particular, it relates to a pharmaceutical formulation comprising at least one helminthic antigen or helminth-specific antibody, which is administered to mammals to prevent and treat allergic disease.

### RELATED APPLICATIONS

The present application is a continuation of international Application No. PCT/US02/16517, filed May 23, 2002, which designates the United States, was published in English, and claims priority under 35 U.S.C. § 119(e) to Provisional Application Nos. 60/316,730 and 60/292,965, filed Aug. 31, 2001 and May 23, 2001, respectively.

### BACKGROUND OF THE INVENTION

1. Field of the Invention

The present invention relates to helminthic compositions in the prevention and treatment of allergies, and methods thereof.

2. Description of the Related Art

Asthma is a chronic lung disease characterized by temporary obstruction of airflow due to inflammation of the bronchial airways. This inflammation further results in increased sensitivity of the airways to a variety of triggers that cause breathing difficulties. (Burt and Knapp, 1996). Asthma sufferers develop symptoms such as dyspnea (difficulty breathing) and wheezing after exposure to allergens and environmental irritants. Indeed, the most common cause of asthma is allergy. Atopic disease, atopy, allergic disease, or as it most commonly referred to as, allergies, is characterized by heightened serum Immunoglobulin E or IgE levels and can be manifested as asthma, allergic rhinitis (hay fever), eczema (dermatitis), and anaphylaxis, to name a few. (Barnes et al., 1999). Among children under the age of sixteen who have asthma, 90 percent of the cases result from allergies. Among asthma sufferers aged thirty and under, approximately 70 percent are allergic. Heredity plays a significant role in the development of allergies. If one parent has allergies, then one in three children will also develop allergies. If both parents have allergies, then all the children will likely have allergies. In the United States, the most common allergy causing allergen is ragweed pollen, an incredibly potent allergy-producing plant. Ragweed is a unique allergen in that it is only found in the United States and it is most often found in unusually high concentrations. Most people who move to the United States are exposed to the ragweed allergen for the first time. Many of these individuals have never had allergies previously in their families and yet develop allergies within two or three years of moving to the United States. Today, allergies are extremely common in urbanized societies and the number of allergy sufferers continues to grow annually.

More than 50 million Americans, more than one out of every five, suffer from allergic disease and allergic diseases are the cause of one out of every eleven office visits to a physician or health care provider (Kaliner, 1991). During the years 1990 and 1992, pollen allergy (hay fever or allergic rhinitis) affected an estimated 10% of, or 26 million, Americans not including those with asthma (Centers for Disease Control and Prevention 1990-92). The number of people currently suffering from allergies is undoubtedly higher considering the increasing prevalence of atopic disease in the world today. The number of people suffering from asthma is equally astounding.

The latest estimates for people suffering from asthma worldwide is approximately 130 million and rising. According to the National Heart, Lung, and Blood Institute, asthma has affected an estimated 14.9 million persons in the U.S., resulting in more than 1.5 million emergency room visits, approximately 500,000 hospitalizations, and more than 5,500 deaths. Furthermore, the prevalence of asthma has been rising since the early 1980s for all age, sex, and racial groups; however, this rise can be seen most markedly among children. Moreover, in 1999, asthma was reported to be the most common chronic disease of childhood, affecting an estimated 4.8 million children. Further emphasizing the detrimental effect asthma has on children is the fact that asthma is the number one cause of school absenteeism among all chronic diseases. Among all diseases, asthma is the number six cause for hospitalization and the number one cause for hospitalization of children. Environmental antigens such as dust and pollens affect a substantial proportion of the population. These individuals, upon exposure, have a specific immune reaction that is somewhat different than the immune response to other pathogens such as bacteria and viruses.

Despite the relative harmlessness of most environmental allergens, exposure to these allergens elicits an immune response that often results in distressing symptoms and bodily damage. This phenomenon, known as allergy or immediate hypersensitivity, is, in essence, immunity gone awry, because the response is inappropriate to the stimulus. Antibody-mediated hypersensitivity responses are usually very rapid in onset and are therefore referred to as immediate hypersensitivity (Vander et al., 1990). In immediate hypersensitivity, exposure to an environmental allergen results in its uptake by antigen presenting cells, which release IL-1 in response. The circulating IL-1 activates B-lymphocytes or B cells and helper T cells or T-h cells. Activated T-h cells, in turn, secrete other lymphokines, specifically IL-2, IL-4, IL-5, IL-6 and IL-13, which stimulate the production of B cell clones, memory B cells, and B cell differentiation into plasma cells, which are essentially the cells responsible for producing (on the order of 1,000 molecules per cell per minute) the antibodies necessary to fight an infection. Memory cells, the cells that mediate active immunity (i.e. resistance to reinfection acquired through exposure to microorganisms, their toxins, or other antigenic material) play an important role when examining the secondary immune response to an antigen with the same specificity. Upon binding to memory B cells, the allergen/antigen in question stimulates the memory B cells to produce more memory B cells and higher affinity antibodies. Their plasma cells manufacture a different profile of antibody classes (in the case of allergic disease, IgE) in much greater quantity, and at a much faster rate (on the order of a few days), when compared to the antibody production rate associated with the primary response (on the order of one to two weeks). Upon re-exposure, the antigen elicits a more powerful antibody response, which in turn constitutes immediate hypersensitivity to the allergen and the eventual emergence of allergy symptoms. The antibodies that are produced in response to allergens are immunoglobulins (Vander et al., 1990).

Immunoglobulins are immunologically active molecules found in the globulin protein fraction of blood serum. The term antibody is often used interchangeably with the term immunoglobulin. Antibodies are complex proteins made up of polypeptide chains and are grouped into five major classes or groups depending on their chain structure, specifically the composition of their heavy chains. All five groups share a basic Y-shaped structure called an antibody monomer. The Y-shaped antibody monomer consists of four polypeptide chains (two identical heavy chains and two identical light chains). The bottom halves of the two heavy chains converge to form the base of the Y, and the top halves form the inside of the two arms of the Y. The light chains make up the outside of the Y arms (see FIG. 1). An antibody binds its complementary antigen at the two antigen binding sites on the ends of the monomer's Y arms. Antigen-binding sites are in the variable regions of the antibody, which contain the unique protein sequences created during lymphocyte differentiation by DNA recombination. The rest of the arms and the base of the Y are the constant region. Because there is an antigen binding-site at the end of each arm of the Y, an antibody monomer is said to have a valence of 2, meaning that it can combine with two epitopes. Each antigen also has a valence, which is the number of antibody molecules with which it can combine (Ingraham, Ingraham 1995). The variable regions give the different immunoglobulins the capacity to recognize and bind with the innumerable antigens for which that particular class of antibodies (e.g. IgE) is responsible for recognizing, binding, and responding to. This gives rise to IgE antibodies that specifically recognize and combine with unique antigens, like those specific to allergens.

The different antibody classes are termed immunoglobulin, or Ig and followed by a letter—G, A, M, D, or E—that is related to the composition of their heavy-chains and the arrangement of their monomer subunits (Ingraham, Ingraham 1995). IgE is a monomer and has the basic shape of a Y, as previously described. IgE constitutes less than 0.01 percent of the body's antibody total and therefore occurs in the body in minute quantities (Ingraham, Ingraham 1995). When the immune system recognizes and responds to an invading foreign protein or allergen, plasma cells respond by releasing IgE specific to the allergen or antigen that elicited the immune response. The IgE antibodies circulate throughout the body and later become attached or fixed to the cell membranes of mast cells and basophils. Mast cells are the cells that produce and secrete the chemicals that result in allergic disease and they are found in every tissue throughout the body. However, they are most heavily concentrated in those tissues that are exposed to the environment (for example; the skin or epidermis, linings of the nose and lungs or mucous membranes, the gastrointestinal tract, and reproductive system). A mast cell has approximately 1,000 histamine containing granules in its cytoplasm, and on its surface are between 100,000 and 1 million receptors/receptor proteins (FceRI) for IgE. Although this may seem like a large number of IgE antibodies fixed to the surface of mast cells, evidence suggests that they are sparsely distributed.

The antibody molecule recognizes the antigen by complexing its antigen binding sites with areas of the antigen termed epitopes. The epitopes fit into the conformational structure of the antigen binding sites of the antibody, enabling the antibody to bind to the antigen. When IgE binds an antigen, it stimulates the mast cell and or basophil to degranulate, releasing powerful inflammatory mediators such as histamine. However, clinical allergy requires efficient cross-linking of high affinity IgE receptors (FceRI) on mast cells and basophils. At least two FceRI-bound IgE molecules must capture a single antigen (bivalent interaction) to induce degranulation and inflammatory mediator release. This is important because it requires that two IgE antibodies of the same antigen specificity be fixed side by side and both must bind antigen to stimulate mast cell degranulation. The importance of this fact will become apparent later, when the effect of competitive inhibition is discussed with regard to preventing mast cell degranulation by saturating mast cell IgE receptor sites with helminthic specific IgE. To effectively prevent mast cell or basophil degranulation, only half of the receptor sites need bind IgE specific to something other than the allergen in question and only every other receptor need be fixed with non-allergen IgE.

The symptoms of antibody-mediated allergy reflect the various effects that histamine and other inflammatory mediators like prostaglandins and leukotrienes have on the body site in which the antigen-IgE-mast cell combination occurs. For example, when a previously sensitized person inhales ragweed pollen, the allergen, or antigen, combines with the IgE antibody that is fixed to the surface of the mast cell's membrane, in the area of the body that is exposed, specifically, the respiratory passages. The inflammatory mediators that are released cause increased mucus secretion, increased blood flow, swelling of respiratory passages, and contraction of the smooth muscle surrounding the airways. The resulting symptoms include congestion, running nose, sneezing, and difficulty breathing and constitute the signs of hay fever. Allergic symptoms may be isolated to the antigens entry site or may become systemic if the mast cell's secreted inflammatory mediators enter the blood stream. If large amounts of the chemicals released by mast cells and or basophils enters the circulatory system, systemic symptoms may result and cause severe hypotension and bronchiolar constriction. This sequence of events can result in anaphylactic shock and possibly death due to circulatory and respiratory failure. Immediate hypersensitivity often progresses to a late-phase reaction lasting many hours or days, during which large numbers of leukocytes, especially eosinophils, migrate into the area. Mast cell mediators released into the affected area attract the eosinophils, which in turn release their own mediators that prolong the inflammation and sensitize the tissues so that less stimulation is needed to evoke a response the next time (Vander et al., 1990). The hypersensitivity response in immune function may be deleterious at times. One might wonder why allergy and asthma prevalence are rising in spite of pharmacotherapeutic improvements; however, when viewed from an evolutionary perspective, this apparent paradox and the emergence of asthma and allergies can be better understood.

One current evolutionary theory with regard to the emergence of asthma and allergic disease in general, proposes that IgE-mediated hypersensitivity stems from an earlier adaptive response to helminthiasis. In the absence of the parasite's antigens (the antigens for which the system arguably may have evolved to combat), the immune system overreacts to common allergens such as pollen and manifests as atopic disease. I, as well as others in the field of evolutionary medicine, propose that the rise in prevalence of allergy and asthma can best be understood from this broader evolutionary perspective and that IgE originated in mammals chronically exposed to helminthic parasites as an adaptive response. Helminths comprise flatworms, roundworms, and flukes. In simple terms, helminths that are parasitic to humans are worms that have evolved to take advantage of an intimate relationship with a human host for survival.

The helminthes include two phyla, the Platyhelminthes or flatworms, and the Nemathelminthes, or roundworms. The Platyhelminthes include tapeworms and flukes, both common human parasites. One example of a tapeworm is the beef tapeworm or Taenia Saginata, which is a flatworm with a segmented body. The beef tapeworm parasitizes humans after they eat infected under-cooked beef. After exposure to or ingestion of the parasite, the larvae migrate to the intestines. The scolex of the worm then attaches to the intestinal wall and the individual is infected. Infection with a tapeworm is a relatively harmless infection. Most people parasitized by one or more tapeworms present no symptoms and are not even aware they are infected. The Nemathelminthes (also called nematodes), or roundworms, have cylindrically shaped bodies and are extremely diverse. About 30 species are parasitic to humans. Some of the more common nematodes include Ascaris lumbricoides, a large roundworm found in the large intestines of infected individuals, Trichinella spiralis, the roundworm that causes trichinosis, and the hookworm, Necator americanus, a worm that parasitizes human hosts in the intestines (Ingraham, Ingraham, 1995).

### SUMMARY OF THE INVENTION

It is an object of several embodiments of the current invention to provide a pharmaceutical formulation for preventing or treating allergies or asthma in a mammal comprising at least one helminth-based agent. A helminth-based agent, as defined herein, shall mean any antigen isolated from one or more species of helminths or any antibody directed to such antigen. Derivatives of such antigens or antibodies, including amino acid fragments or synthetic, chemically modified or substituted fragments are also included within this definition. The helminth-based agent is capable of ameliorating the allergic reaction to a wide range of antigens/allergens. In many embodiments, a pharmaceutically acceptable compound, including an adjuvant, carrier and/or diluent, is administered in conjunction with the helminth-based agent. The formulation may be in a variety of forms, including injectable fluids, suppositories, powder, tablets, capsules, syrups, suspensions, liquids and elixirs.

In another aspect of this invention, an immunogenic amount of a helminthic antigen (either the protein, glycoprotein, or any of the other forms that an helminth secreted/produced immunogenic antigen may assume) is administered. In a preferred embodiment, the antigen is isolated from 3-5 different nematodes, trematodes and/or cestodes. Preferably, the antigen is isolated from Capillaria hepatica and/or Dicrocoelium dendtriticum and/or Schistosomes.

In another aspect of the current invention, an effective amount of a nucleic acid molecule encoding at least one relevant epitope of a helminthic organism is administered to prevent or treat the allergic reaction. In another embodiment, a recombinant cell transformed with a nucleic acid molecule encoding a helminthic protein is used.

In one aspect of this invention, the helminth-based agent is an antibody directed to at least one epitope of a helminthic antigen. Preferably, the antibody is a monoclonal antibody.

It is another object of several embodiments of the current invention to provide a pharmaceutical composition for preventing allergies or asthma in a mammal comprising a helminth-based agent in an amount sufficient to regulate IgE. In one embodiment, the pharmaceutical formulation is administered by a route which results in systemic absorption of an immunogenic amount of the helminth-based agent. Preferably, the formulation is administered intradermally, subcutaneously, intravenously, orally or rectally. One of skill in the art will understand that, in accordance with various embodiments of this invention, the formulation can be made and administered in the same manner as traditional vaccines are currently used.

In a preferred embodiment, this invention provides a method of treating and immunizing a human against IgE-regulated allergic reactions by administering an effective dose of a helminth-based agent to a human during any stage of his or her life, preferably as early in life as possible, optimally, immediately following birth.

In one embodiment, the dose is determined by measuring total serum IgE levels and serum levels of IgE specific to allergens, and calculating the desired level. In a preferred embodiment, the desired level is greater than about 1500 IU/ml, preferably about 3000 IU/ml.

### BRIEF DESCRIPTION OF THE DRAWINGS

FIG. 1 represents an antibody monomer. All antibodies have a basic Y-shaped structure called a monomer. The tail and the inside of the Y's arms are made up of heavy chains, and the outsides of the Y's arms are made up of light chains. At the end of each arm, in the variable regions, the monomer has an antigen-binding site. Sites for binding to phagocytes and complement (C1) are in the constant regions.

FIG. 2 illustrates the relationship between total blood serum IgE levels and the mean house dust and mold specific IgE levels for 27 patients, ages 13-67 (data from Lynch et al. 1985: Table 1).

FIG. 3 shows cutaneous reactions and humoral antibody levels in the study group.

FIG. 4 shows immediate hypersensitivity skin test and RAST positivity in Amazonian Indians.

FIG. 5 represents helminthic infection detected in serially collected feces samples of 114 Amazon Indians.

### DETAILED DESCRIPTION OF THE PREFERRED EMBODIMENT

In the United States, as well as most Western societies, helminthic infestation is uncommon, if not rare. This is a fairly recent occurrence in the evolution of humans when examined from an evolutionary viewpoint. The human immune system evolved long before the emergence of humans and independently of their cultural ways. Of particular interest are the antibodies or immunoglobulins. The five classes of immunoglobulins or Ig's are present in all placental mammals. IgE is believed to have emerged most recently, evolving within the past 300 million years. Given the recent emergence of modern humans (˜100,000 years ago) and the very recent elimination of parasitic worms from Western society (˜50-100 years ago), the complete elimination of such a fundamental process in the immune system, even if it occasionally overreacts, seems very unlikely in such a short period of time (Barnes et al., 1999). An examination of our evolutionary past is necessary in order to put into context the co-evolution of humans, helminths, and the human immune system, as well as the interaction among the three.

Although not wishing to be bound by the following theory, it is believed that the co-evolution of humans, helminths, and the human immune system contributes to the ability to utilize heminth-based antigens to prevent and/or treat allergies and asthma in humans. More specifically, as early hominids expanded their niche and the variety of foods they consumed, they became hosts to increasing numbers of helminths. Indeed, humans are, in fact, definitive, intermediate, or accidental hosts for more than 70 species of helminths. The shift from a hunter and gathering way of subsistence to an agriculturally based existence led to an increase in sedentism and with it came an increase in parasitic disease spread by contact with animals, other humans, and their waste. The pronounced relationship between humans and parasites continued until acute infectious diseases were controlled, particularly in industrialized societies, which led to a rise in chronic, noninfectious, degenerative diseases. Allergy is just one example of a chronic disease. Because parasitic infections are naturally endemic in all species including humans, it is probable that allergies and asthma are a problem arising in recent times and one that is limited to mammals that have drastically reduced their exposure to parasites over the past century. It appears that the allergy and asthma phenotype is the price humans have had to pay for getting ahead in the host-pathogen arms race in human history (Barnes et al., 1999; Hurtado, 1999).

In industrialized societies, helminths (e.g., flatworms and roundworms) have been virtually eliminated due to advancements in food and water quality, living conditions, education, medicine, and, in general, the transformation of Western society from hunters and gatherers to an industrial based society. With this in mind, it is necessary to understand that if the selective pressure for a specific adaptive feature is reduced, natural selection will not necessarily lead to a quick degeneration of or noticeable change in the system (Barnes, 1999). This being the case, modern American or westernized immune systems have not had enough time, in evolutionary terms, to adapt and/or evolve to the decrease in helminthic parasite load, resulting in the rise in prevalence of allergic disease.

The degree of intestinal helminthiasis is related to the expression of allergy and/or asthma symptoms or atopic disease. There is a lower prevalence of atopic disease in less developed countries when compared to industrialized societies. There is also a distinct difference between the prevalence of atopy in rural areas when compared to those observed in urban areas within the same country. Examination of the environmental changes that have occurred over the years in contemporary populations and the prevalence of allergic disease among them, clearly illustrates that populations in under-developed regions, with a high prevalence of helminthiasis, have a lower prevalence of asthma and allergic disease. Numerous studies have examined the various environmental factors (such as exposure to indoor allergens, pollution, or change in diet) that might explain this rise in atopy; however, there is little consistent evidence to suggest that the changes in environmental conditions could account for the rise in atopic disease (Yazdanbakhsh et al., 2002). Populations who reside in societies who have virtually eliminated the chance of helminthic infection have suffered an obvious increase in the prevalence of chronic disease.

A recent theory attempting to explain this phenomenon proposes that a lack of intense infections in industrialized countries, due to increased use of antibiotics, implementation of vaccines, and improved hygiene, alters the human immune system in such a way that it over responds to innocuous substances. This theory is commonly referred to as the “hygiene hypothesis”. Experts in the field of allergic disease are now examining the connection between “growing up too clean” and the resulting overreaction of the immune system to common allergy inducing substances.

The most recent hypothesis linking helminthiasis with decreased atopy proposes an alternative immunological framework for the “hygiene hypothesis”. The immunological framework on which the hygiene hypothesis bases its conclusions asserts that bacterial and viral infections early in life direct the developing immune system toward a strong T-h1 imprinting, counterbalancing the proallergic responses of T-h2 cells. The hygiene hypothesis asserts that an overall reduction in microbial burden will result in an underdeveloped or weak T-h1 imprinting, leading to unrestrained T-h2 responses, resulting in atopy. This assertion is contradicted by the fact that T-h1 autoimmune diseases, like type 1 diabetes, are increasing and that T-h2 skewed helminth infections are not associated with allergy. Furthermore, when one considers that more than one billion people worldwide are heavily parasitized by helminths and are rarely afflicted by allergic disease, then it becomes clear that a strong T-h2 response is not the precipitating factor in an allergic response. Moreover, with regard to helminth parasites, it has been shown that asymptomatic infections are correlated with high levels of another T-h2 dependent isotype, IgG4, further demonstrating the flaws associated with tying a strong T-h2 response with the development of allergy. Interestingly, parasite-specific IgG4 antibodies can inhibit IgE mediated degranulation of effector cells (Yazdanbakhsh et al., 2002).

A high prevalence of chronic and acute infections in developing countries results in persistent immune challenge. Bacteria, viruses, and helminths (the microbes responsible for continuous immune system challenge) carry distinct signature molecules that interact with dendritic cells (dendritic cells direct T cell differentiation) to stimulate T-h1 type and T-h2 type immune responses, which, when uncontrolled, lead to autoimmunity and allergy. High pathogen burden may result in the accumulation of novel signature molecules that endow dendritic cells with the ability to induce regulatory T cells. Regulatory T cells produce suppressory cytokines and are an integral part of an anti-inflammatory network that ensures that inflammatory T cells (both T-h1 and T-h2) and their downstream effectors are kept under control. It is likely that such a network would be underdeveloped in industrialized countries with a low pathogen load, allowing more inappropriate immunopathological reactions to develop more readily. Down regulatory molecules such as Interleukin-10 (IL-10) and transforming growth factor beta (TGF-B) have been implicated in this immunosuppression and high levels of IL-10 are commonly seen in individuals chronically infected with helminths (Yazdanbakhsh et al., 2002).

As previously noted, allergies are inflammatory diseases dependent on T-h2 type responses and initiated by mast cell degranulation. The release of the subsequent inflammatory mediators accompanies the production of pro-inflammatory cytokines. Therefore, the presence of a strong anti-inflammatory regulatory network, characterized by elevated levels of L-10 and TGB-B, produced and secreted by antigen-presenting cells and/or regulatory T cells, could help to prevent the series of events leading to allergic inflammation. IL-10 can inhibit mast cell degranulation, providing a mechanism for the observed negative associations between IL-10 and atopic disease. Indeed, the induction of an anti-inflammatory network by persistent immune challenge provides an explanation for the inverse association of many infections, particularly helminthiasis, with allergic disorders. Moreover, since parasites are often long-lived and parasitize human hosts for prolonged periods of time, it is not surprising that they possess modulatory molecules that ameliorate host responses in order to enhance their survival, as well as the survival of their host (Yazdanbakhsh et al., 2002).

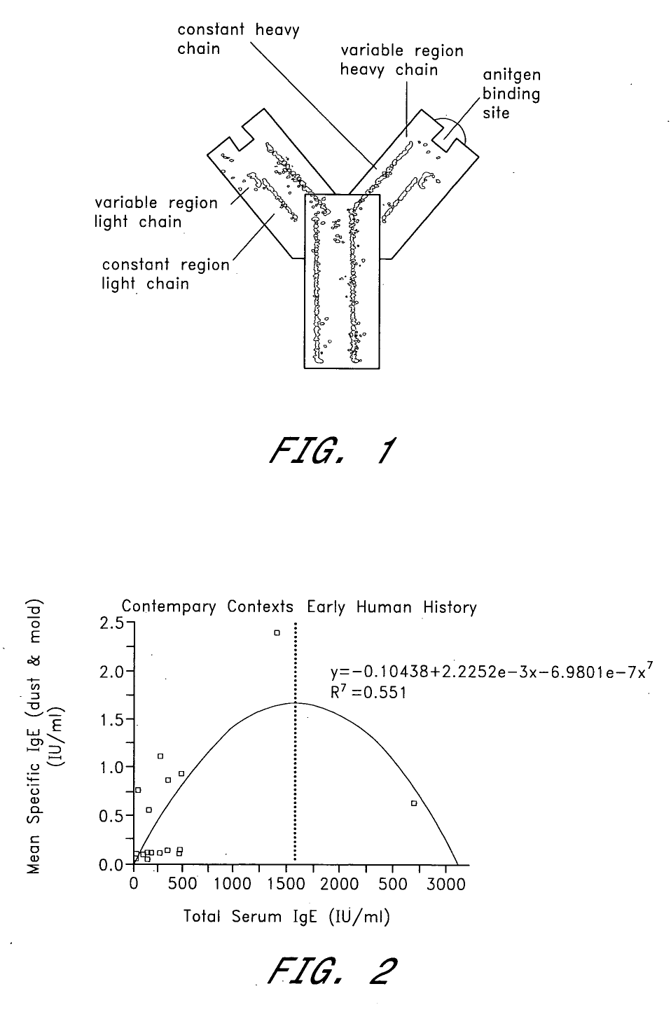

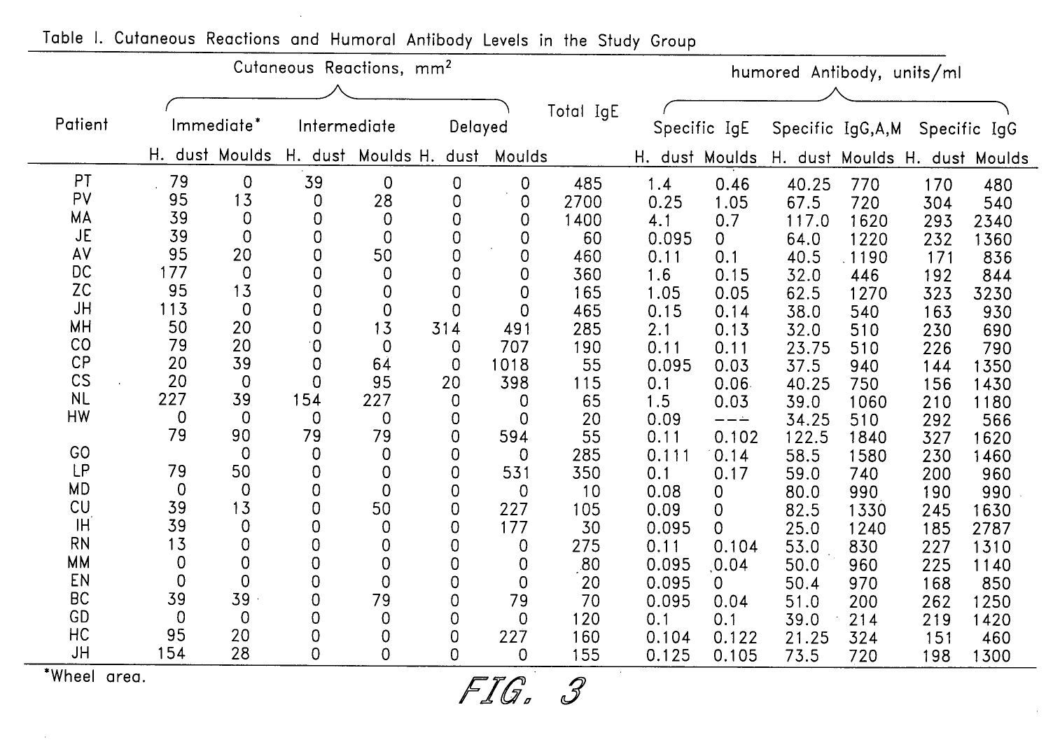

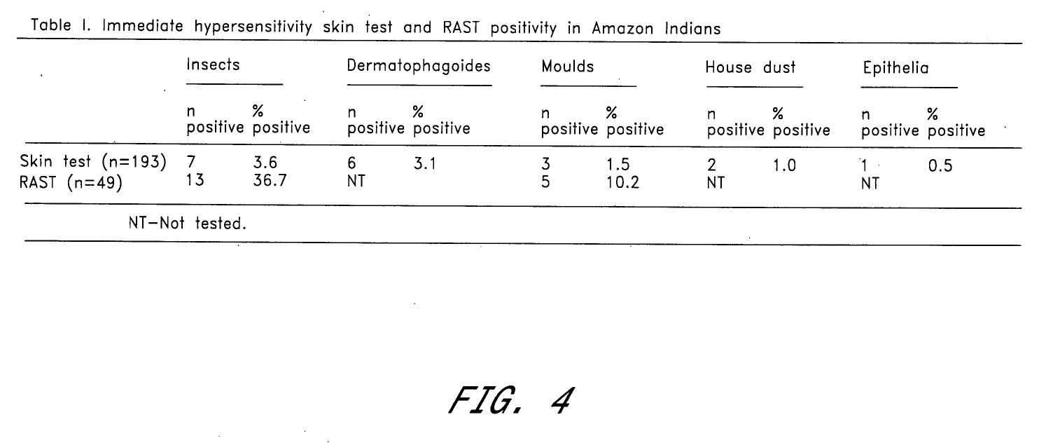

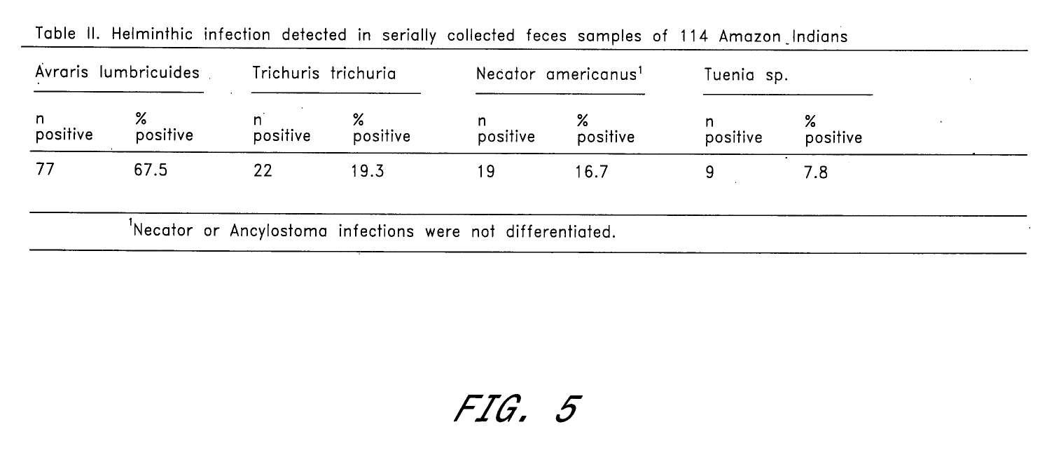

Another theory that may explain this phenomenon proposes that antibodies normally involved in combating helminthic infestations do so at such a rate that there is an almost complete saturation of these antibodies in the tissues and plasma of humans, to the degree that there is little, if any, response to innocuous allergens. However, in the absence of such systemic IgE saturation, the immune system is free to respond and over-react to common allergens (Barnes 1999). IgE circulates in serum at varying levels (range: 0.05 IU/ml-46,850 IU/ml). A portion of the IgE reacts specifically with identifiable environmental antigens (allergen-specific IgE); whereas, another portion of IgE is directed against other non-allergenic antigens, like those specific to helminths or helminth-specific IgE. When animal and human mast cells become saturated they fail to express the allergen specific response as, for example, when all or most available receptors are occupied by other IgE antibodies with a different specificity. For example, among individuals heavily parasitized by helminths, which also are free of asthma and show high total IgE levels, it has been established that allergic reactions of the skin are not expressed. Thus, although specific antiallergen IgE may be present in these individuals, it does not react functionally in the presence of high loads of unrelated IgE (Hurtado et al., 1999). The mast cell saturation principle provides insight into understanding reaction norms between helminthic load, allergen exposure, and the expression of allergic disease. One way to examine these norms is to compare total serum IgE levels, as determined by parasite load, and allergen-specific IgE. The mast cell saturation theory should predict an inverse relationship between total IgE (specific to helminths) and allergen-specific IgE. For example, as total IgE serum levels increase, mast cell bound IgE receptors should become saturated, impeding allergen-specific IgE from accessing these receptor sites. However, the interrelationship between IgE antibodies is more complex. Effective competitive inhibition of allergen-specific IgE seems to only occur at high levels of parasite load and not at intermediate levels. At intermediate levels, serum IgE synthesis appears to be large enough to substantially increase total serum IgE levels, but not large enough to block mast cell activation by allergen-specific antigens. Allergen specific IgE positively corresponds with total IgE up to a point (˜1500 IU/ml), but beyond that point it appears to be negatively correlated (Hurtado et al., 1999) (see Table 1 and FIG. 2).

The relationship of high loads of intestinal parasites and a very low incidence of atopic disease among Amerindians of the Amazon basin confirms the principle of mast cell saturation due to high levels of helminth-specific IgE (Lynch et al., 1983). This research supports the proposition that thresholds greater than about 1500 IU/ml total IgE may be typical of our evolutionary past. Indeed, the typical Amazonian's helminthic load is extremely high, ranging between 60% and 98% prevalence for individuals of all ages (Hurtado et al., 1999). Lynch and his associates obtained their results by examining 274 unselected Amerindians with 55.8% of the subjects being female and all subjects being 18.5±13.6 years. Prick skin tests were performed on each of them with extracts of Dermatophagoides sp, Ascaris Lumbricoides, five common molds, six types of insects, skin cells (epithelia) from 8 animal species and 23 local foods. The frequency of positivity in immediate hypersensitivity skin tests was found to be extremely low (6.7%). Because sera from a significant proportion of the study group contained specific IgE antibodies against the test allergens, and their histamine-induced skin responses were normal, these results support the proposition of an inhibited expression of allergic reactivity in such populations. The intense helminthic infections and the extremely high total serum IgE levels measured (geometric mean 13,088 IU/ml) indicate the probable occurrence of mast cell saturation by parasite-induced IgE. Allergic disease occurs in these individuals at a particularly low frequency. In fact, of the total 75,013 medical consultations conducted in the study area by the Venezuelan Health Ministry over a recent 5-year period, only 1.02% was for asthma and 0.59% for other allergic conditions (Lynch et al., 1983).

These facts may give us our best insight into what the parasite prevalence rate was throughout human history. Total IgE levels in these populations are the highest ever measured in contemporary populations (11,975 IU/ml and 13,088 IU/ml among the Waorani and various Venezuelan Amazon groups, respectively, see table 2), and asthma and allergy rates are extremely low or nonexistent (Hurtado et al., 1999). Other extant populations residing in rural areas also typically show IgE levels above the 1500 IU level, and low rates of allergic disease and/or low percentage of positive skin tests. Proponents of the alternative immunological framework for the hygiene hypothesis dismiss the mass cell saturation theory and attempt to explain its flaws by taking issue with the methodology employed by some of its researchers. Moreover, they point out that recent studies have shown that the FceRI numbers on mast cells respond to the concentration of circulating IgE by changing receptor concentration (increasing the concentration of receptors in response to high levels of circulating IgE) and accommodating additional binding (Yazdanbakhsh et al., 2002). However, if one assumes that mast cells do increase receptor concentration in response to high IgE levels or high helminth specific IgE, it stands to reason, that if there is a much greater plasma concentration of helminth specific IgE compared to that of allergen specific IgE, then the odds are statistically much higher that a helminth specific IgE antibody will fix to the newly created binding sites on the affected mast cells or basophils and not the underrepresented, outnumbered, competitively inhibited allergen specific IgE. Whatever hypothesis one chooses to subscribe to, the end result is the same. Helminth infections are inversely correlated with allergies.

Parasite load may not only play a role in inhibiting production of IgE specific to allergens and blocking allergen specific IgE from binding to mast cell surface receptors, but also the timing of exposure during growth and development may be critical, because the probability that the asthma or allergy phenotype will be expressed in an individual is negatively associated with the age at which a person is exposed to common allergens. Moreover, experimental studies with rats show that exposure to environmental allergens before exposure to helminths is positively correlated with the probability of developing allergic disease. South American Indian children tend to be exposed to and infested with parasites at a very young age. However, they are exposed to low levels of indoor allergens due to the fact that they live in well ventilated huts and use traditional bedding, which is a poor breeding site for dust mites, unlike the bedding or mattresses utilized by modern society. Because Western society tends to live in less ventilated housing and spends more time indoors, the risk of exposure to indoor allergens just after birth is extremely high. Consequently, decreases in parasite load and the introduction of insulated housing and Western bedding in recent human history help explain why total IgE levels <1500 IU/ml are characteristic of populations residing in metropolitan areas throughout the world today (Hurtado et al., 1999). The importance of early exposure to helminths or their associated antigens is further exemplified by the fact that mast cells can last for years.

Most antibodies last in the body approximately three weeks; however, IgE can fix to its receptor on mast cells for years. An example illustrating IgE's long-lived binding ability can be found by taking note of the fact that most people who have had an adverse allergic reaction to penicillin as a child are still allergic to the drug as an adult. The IgE antibody that was created as a 6-year-old child would still be present in the 40-year-old adult. It is fixed to the mast cells, which are long-lived cells, and it conveys incredibly long-lived sensitivity to allergens. In one research study, it was found that after releasing histamine from its granules, a mast cell can regenerate all of the granules lost by secretion and once again do damage to the person the next day when he or she is re-exposed to the allergen that initiated the immune response.

This fact and all the previously mentioned attributes of the IgE/mast cell/antigenic immune response are important in that they are necessary knowledge when it comes to formulating a possible treatment, drug, or vaccination in the battle against allergies and asthma. Since high helminthic load in humans' results in a decrease in atopy, it would be desirable to simulate the effect that high levels of intestinal helminths have on the human immune system. In one embodiment of the present invention, the effect of high levels of intestinal helminths is stimulated, thus overcoming the disadvantage of direct infection with an helminthic parasite.

In a preferred embodiment of the present invention, the production of helminthic-specific IgE is initiated by administering the protein antigen specific to one or more helminths. The aforementioned antigen is isolated and collected from at least one species of helminth, preferably 3-5. The antigen is extracted from the organism(s) at any stage of development (cercariae, larval, adult worm etc.) and can be isolated from any helminth, including those that don't normally parasitize humans; however, it is unknown if the body will have an immune response to an antigen it has never before encountered or if the immunogenicity of the previously mentioned antigens will produce a strong enough response to produce the desired effect.

In a preferred embodiment, administration of the helminths antigen is accomplished by administering the protein antigen in pharmaceutical compositions adapted for systemic administration. In various embodiments, the pharmaceutical composition is in the form of an injectable fluid, suppository, powder, tablet, capsule, syrup or elixir. In several embodiments, pharmacologically acceptable adjuvants, diluents, carriers, lubricants and the like are administered in conjunction with the antigen.

Further, one skilled in the art will understand that the amount of the antigen used in the current invention will depend on the dose required for administration and the treatment desired. One skilled in the art will appreciate that “treatment” refers to any desired purpose for administering the antigen, including prevention, control, cure, maintenance, or improvement of allergies or asthma. By varying the concentration of the ingredients, size, number and/or amount of tablets, capsules, suspension or liquid, a wide range of doses may be administered.

One skilled in the art will also appreciate that the current invention is not limited to the delivery of a single antigenic agent. Although a single type of antigen may be administered, more than one agent may be delivered simultaneously using various embodiments of the current invention. For example, in one administration, the recipient may receive a combination of two or more antigens.

In a preferred embodiment, the pharmaceutical composition is administered intradermally. However, the confection can be taken orally or rectally, because most of the worms that parasitize the gut synthesize and release their highly antigenic secretions into their immediate environment, where they are readily absorbed, resulting in a strong immune response in their host (Olgilvie et al., 1981). The antigens secreted on the endothelium of the gut can readily pass into and through the intestinal mucosa, due to the relatively small size of the helminths antigenic protein.

One skilled in the art will understand that other methods of drug administration may also be used. In one embodiment, the pharmaceutical composition is intravenously administered. Preferably, the compound would be prepared as a solution or suspension capable of being administered by injection. In certain embodiments, it would be useful to formulate these compounds in suppository form. Once the helminthic antigen(s) are systemic, they would act upon the immune system just as if a worm was parasitizing the subject. This would produce the desired effect of mast cell saturation or production and secretion of antiinflammatory mediators like IL-10 that prevent and diminish the immune response to innocuous substances, such as those responsible for allergies and asthma. It should also be administered as early as possible after birth in order to minimize the patient's exposure to allergens before inoculation and the resulting competition for available mast cell surface receptors.

In one embodiment of the current invention, the initial dose of the confection is followed-up with blood tests to measure total serum IgE (helminthic-specific) and serum levels of IgE specific to allergens, by employing assays, including, but not limited to the enzyme-linked immunosorbent assay (ELISA), and the like.

In another embodiment, after determination of the patient's IgE levels, an appropriate dose is administered based on the patients' measured IgE levels and the desired level of greater than approximately 1500 IU/ml, preferably >3000 IU/ml.

In one embodiment, a particular helminthic antigen is administered at least once. In several embodiments, when the antigen is administered a second time, some of this antigen will bind to fixed helminthic-specific IgE on the surface of mast cells and will stimulate them to degranulate and initiate an inflammatory response; however, the immune response will be hardly noticeable with regard to symptoms.

In a preferred embodiment, simulation of helminthic infestation involves direct injection of helminth-specific IgE antibodies. Preferably, monoclonal antibodies are used. Several methods of the current invention, which involve administration of helminth-specific antibodies, are particularly advantageous because they would not require the immune system to produce the antibodies itself. As such, the immune system would not be taxed, thereby leaving it intact to combat other invading microbes or antigens.

A particular embodiment of the current invention involves direct injection of prepared monoclonal IgE antibodies specific to helminths, utilizing the process previously described for preparing intravenous confections. This embodiment is particularly well suited for the elderly and immuno-compromised individuals who should not have their immune systems taxed further.

By referring to FIG. 2, one can see that the expression of the asthma/allergy phenotype is minimized at blood serum levels of IgE >3,000 IU/ml. In one embodiment of the present invention, the recommended amount of total serum IgE is approximately between 500 IU/ml and 50,000 IU/ml, preferably between 3000 IU/ml and 15,000 IU/ml. The preferred maximum is based upon the highest level of total serum IgE measured in an extant population.

In various aspects of this invention, the quantities of IgE specific antibodies or helminth specific antigen given by vaccination reflect the necessary number of molecules of the specific IgE antibodies or antigen molecules and would constitute the major constituent of the vaccination. In a preferred embodiment, the vaccination would be prepared as previously described, just following birth, or the first possible opportunity after delivery, possibly in conjunction with other typical immunizations that a newborn might receive. In several embodiments, the IgE levels, both total and specific, would be measured by ELISA on a regular basis (˜1-6 months after vaccination and every 3-6 months thereafter), and follow-up inoculations would be given if the level of total serum IgE drops near or below the recommended minimum of approximately 3,000 IU. The dose of the follow-up inoculation may be determined by several methods, including, but not limited to, examining the current total serum IgE level and comparing that value to the desired value and administering the appropriate dose so as to achieve the desired level of total serum IgE.

In several embodiments, the helminth-specific antibodies are manufactured or synthesized in the laboratory. Preferably, monoclonal antibody technology is used. Monoclonal antibodies can be genetically engineered with relative ease. Indeed, new recombinant DNA techniques for manufacturing monoclonal antibodies mimics in a laboratory setting exactly what the immune system would do itself in the human body (Ingraham and Ingraham 1995). Monoclonal antibodies can be synthesized using standard techniques and methodology well known in the art (Harlow & Lane's Antibodies).

Generally, the production of monoclonal antibodies is accomplished by inserting a DNA fragment that contains the gene of interest into the purified DNA genome of a self-replicating microorganism, usually a bacterium, virus, or plasmid. Human antibody genes can then be packaged in bacteriophages and introduced into Escherichia coli, which then produces human monoclonal antibodies. A person's entire antibody repertoire can now be genetically engineered into a culture of bacteria. Furthermore, the normal replication mechanisms of the phage can produce more than a trillion identical phage DNA molecules in less than a day, thereby amplifying the amount of the inserted human DNA fragment by the same factor. Later, the bacterial cells that produce the desired antibody are identified and isolated for further manipulation and extraction of the desired monoclonal antibody.

Another way of cloning specific DNA sequences rapidly without the need for a living cell involves a fairly recent technique called the polymerase chain reaction or simply PCR. PCR allows the DNA from a selected region of a genome to be amplified a billion fold and is done so in a series of steps or cycles where the primary reactants are the DNA segment, a purified DNA polymerase, and a chemically synthesized DNA oligonucleotide. When combined and utilized properly, the end result is the production of virtually limitless amounts of the desired cloned antibody. With regard to various aspects of the current invention, monoclonal antibodies can be synthesized using the aforementioned techniques, or using various techniques and methodology known in the art.

Several embodiments of the current invention are particularly valuable because an injection of helminthic-specific IgE antibodies would not tax the immune system. First, the immune system would not have to produce the antibodies itself like it would if the patient was administered the helminth-specific antigen. Furthermore, when follow-up inoculations are required, another injection of the specific monoclonal antibodies would not stimulate mast cells to secrete their inflammatory mediators, as would be the case when a follow-up dosage consisted of the helminthic-specific antigen. Moreover, if a particular worm species, or more than one species, were chosen that was not endemic in the United States, then the possibility of exposure or infestation is kept to an absolute minimum and a possible subsequent immune response to the species of worm(s) is virtually eliminated.

Although any helminth-specific antigen or antibody can and would produce the desired result, it would be best to choose one that rarely occurs, if at all, in the United States or better yet, worldwide and one that is highly immunogenic, thereby producing a strong immune response. In a preferred embodiment of the current invention, a species of worm that is as rare as possible is used, enabling the vaccine containing either the antigenic material or the IgE antibodies specific to this particular worm antigen, to be utilized in a vaccine worldwide.

There are at least 70 species of parasitic worms known to infect man. Several of these are rare or accidental and of little consequence to a mankind as a whole (Crompton and Joyner, 1980). In many embodiments, at least one type of helminth-specific antigen or monoclonal antibody is used for vaccination purposes. In a preferred embodiment, the antigen or antibody for vaccination is derived from the nematode Capillaria hepatica. C. hepatica mainly parasitizes rodents, although dogs, squirrels, and monkeys can become infected as well. Human infection is very rare and only a few cases have been reported (Spencer and Lee, 1977). In fact, there were only 10 reports of human infection with C. hepatica before 1971. (Beck and Barrett-Conner, 1971). C. hepatica is rarely found parasitizing humans today as well. In several embodiments, the rarity of C. hepatica makes it one of the two preferred choices for the aforementioned vaccine. In another embodiment, the trematode Dicrocoelium dendtriticum, more commonly known as a liver fluke, is utilized. D. dendtriticum is a common parasite that is found primarily in sheep.

In many embodiments, more than one type of helminthic antigen or helminth-specific monoclonal antibody is used for vaccination purposes. In one embodiment, the antigen or antibody for vaccination can derived from the nematode Loa loa that infects the skin and the eyes. Loaiasis, the disease caused by Loa loa, and the parasites themselves are only found in Africa and therefore would be useful in a vaccine everywhere, except in the continent of Africa. In another embodiment, a candidate for an helminth-specific antigen or monoclonal antibody to be used for a vaccine are the schistosomes, a type of trematode or flatworm that causes the disease schistosomiasis. Schistosomes are prevalent in Asia, Africa, and the Middle East. Although schistosomiasis is seen in the United States among immigrants from endemic countries, the infection can never become endemic in North America because the essential snail hosts are not found here (Ingraham, Ingraham, 1995). Therefore, the antigen extracted from the Schistosomes or the antibodies that are produced upon exposure to the worm(s) antigen(s) could be ideally utilized in a vaccine everywhere, except Asia, Africa, and the Middle East. Children born in the United States and other westernized societies would not likely be infected by these parasites, unless they traveled to the country or countries in which the aforementioned helminths are endemic and they encountered the parasite in the stage of development that corresponds to human susceptibility to infection. When allergy vaccinated people travel to these countries, appropriate measures to prevent contact and infestation with these parasites may be taken. Further, the individual could further protect himself or herself by being tested, after his or her trip, to determine if he or she was exposed to the helminth in question. Indeed, any individual may be tested for the presence or evidence of infestation by the helminth in question, assuming the vaccine does not utilize the antigen, or monoclonal antibodies specific to the antigenic protein, from one of the aforementioned preferred choices, specifically, C. hepatica and/or D. dendtriticum.

One advantage of several embodiments of the present invention is the fact that the vaccine would not only be beneficial to newborns, but all people currently living in America or other Westernized societies, if not the entire world. The benefit of the treatment may take time to realize in adults considering the fact that mast cells can last for years and the replacement of lost mast cells and their subsequent saturation by helminthic-specific IgE (due to competitive inhibition of the out-numbered allergen-specific antibodies) can take years. However, the only currently available immunological method to reduce allergy symptoms is the “allergy shot”. IgG, an immunoglobulin or antibody that defends tissues from systemic infections, is the major serum antibody in mammals. One of the functions of IgG is the down regulation of IgE production and IgG is often referred to as a blocking antibody. Immunotherapy (allergy shots) stimulates the production of IgG to ensure this blocking action (Hurtado et al., 1999). Among individuals exposed to the same level of allergens and having the same levels of total serum IgE, an allergy shot or IgG may prevent allergic symptoms entirely, somewhat, or not at all. The reasons IgG fails to provide its blocking function in some individuals and under some conditions is probably due to the fact that total IgE production increases under conditions when IgG is being used to fight other concurrent problems, such as bacterial infections, and the differences reflect these physiological trade-offs (Hurtado et al., 1999).

U.S. Pat. Nos. 5,911,988, 5,908,839, 5,843,441, 5,780,481 and 5,560,915 represent advancements in the field of allergy and/or asthma treatment. One major disadvantage of drugs and treatments currently utilized or proposed is that they seek to reduce the symptoms of allergy and asthma and do nothing to prevent their expression. Moreover, allergy shots are utilized after one begins to present symptoms of allergic disease and it can take years to reverse the expression of allergen-specific IgE induced symptoms. Yet another disadvantage to allergy shots, as well as many of the current proposed vaccines against allergy, is that they seek to control the expression of allergic disease by eliminating all IgE from one's system. This method of controlling atopic disease is flawed, in that it fails to recognize or consider that removal of all IgE would result in the bodies inability to mount an immune response against mold or fungal infections, which are common, not to mention the body's inability to combat helminths. Mold, fungi, and helminth infections or infestations are all combated and controlled by the same antibody that reacts to common allergens, that being IgE. In general, combating allergic disease and its accompanying symptoms by removing IgE from the human immune system's arsenal is a flawed idea at best. Interestingly, it has been found that high levels of serum IgE have tremendous health benefits for those affected. Additionally, high levels of serum IgE are more representative of our evolutionary past and inducing such a state would represent a return of our human immune system to a previously, tried and true, homeostatic state; whereas, decreasing or eliminating IgE introduces a new unknown state into the human immune system. Therefore, several embodiments of the current invention are particularly useful because they provide a pharmaceutical composition or vaccination for the prevention of atopic disease without introducing an unknown state into the human immune system, which would result in unknown and possibly detrimental consequences. In a preferred embodiment, treatments or vaccines of the current invention, with the major constituent being helminth-specific antigens or monoclonal antibodies, will prevent the symptoms of allergies or allergic disease from ever occurring. The list of who would benefit from this treatment/vaccination is indeed a long one.

In one embodiment, this invention is targeted to those who currently suffer from, or those yet to be born who are genetically predisposed to, allergies and or asthma. Although heredity does not explain the reason for all who present allergic disease, it is a very influential determinant. It is envisioned that various aspects of this invention will be used to vaccinate all newborns. The benefit of vaccinating all newborns is the protection bestowed upon the substantial number of people not genetically predisposed to allergy, who would not be vaccinated if the caveat of predisposition were attached. Furthermore, if all newborns are inoculated, they too will profit from the beneficial side effects of this treatment/vaccine (the beneficial side effects to be discussed in detail later). Not only do patients and their familys greatly benefit, but also the nation as a whole would save billions of dollars annually in medical costs associated with hospital and office visits, treatments, and medications. Furthermore, the tremendous burden on the economy that stems from lost school and work days would be greatly reduced. According to the New England Journal of Medicine, asthma treatment cost an estimated $6.2 billion in 1990, with 43% of that total cost being associated with emergency room use, hospitalization, and death. Loss of school days alone resulted in significantly decreased productivity that cost an estimated $1 billion (Weiss, 1992). In 1998 alone, the direct and indirect costs of the disease totaled $11.3 billion. Hospitalization accounted for the single largest cost, that being $3.6 billion (National Heart, Lung, and Blood Institute, 1999). Seven out of 10 asthma visits (68.7 percent) resulted in a scheduled return visit and the vast majority of asthma visits had medication prescribed. There were about 56.8 million drug mentions (a drug mention being the physicians entry on the Patient Record) at asthma visits during 1993-1994, an average of 28.4 million mentions per year (Burt et al., 1996).

Several embodiments of the current invention offer particular economic advantages. Potential allergy sufferers are not the only ones who will benefit. Doctors will benefit by having increased time to spend with other patients. Medical insurance companies and governmentally owned and operated medical insurance agencies will benefit from a substantial decrease in monies expended on the treatment of allergies and asthma, thereby resulting in lower insurance costs and fewer tax dollars necessary to fund and operate government health insurance agencies. The lower taxes and the decrease in medical insurance premiums will be of great benefit to all Americans. The manufacturer of the pharmaceutical composition will profit tremendously, with the added security of knowing that they can't be sued in the few instances where there might be adverse or detrimental effects. This law enacted by Congress benefits us all, because the benefits of a treatment/vaccine, like the one described, far outweigh the few, if any, possible negative reactions or side effects that may occur. Patients other than those with allergic disease will benefit as well, because total serum IgE also appears to play a positive role in other debilitating or life-threatening conditions. For example, high serum IgE levels decrease the risk of sudden cardiac death following a myocardial infarction (IgE depresses clot formation), slow the development of cancerous tumors (IgE decreases tumor growth and incidence), and decrease the risk of death during traumatic injury (IgE is inversely associated with organ failure and mortality in trauma patients) (Hurtado et al., 1999). A revealing study about the benefits of high levels of serum IgE and the effect it has on the prevalence of sudden cardiac death following an acute myocardial infarction (AMI), was conducted. Mean levels of serum IgE were significantly higher in the group of patients without sudden cardiac arrest than in the group of patients who suffered this complication. Furthermore, none of the patients with an IgE level higher than 400 IU/ml experienced sudden cardiac arrest; whereas, of the patients who experienced sudden cardiac arrest, only 5% of the patients had an IgE level higher than 200 IU/ml. High serum IgE levels were also found to be associated with delayed thrombin generation in the clotting blood of AMI patients. In these patients, the building up of a clot inside a critically narrowed coronary artery was delayed because of the late appearance of thrombin. It appears that patients with high serum IgE levels are protected against sudden cardiac death through the depression of clot formation and this slowing of clot formation is due to the late appearance of thrombin. It appears that intestinal helminthiasis provides humans with a previously unrecognized inhibiting quality, with regard to allergens, and this ability to inhibit allergen sensitivity was and is a result of natural selection and the evolution of the human immune system. Moreover, it appears that high serum levels of IgE (which in the past were induced by prevalent helminthiasis) also afford people a previously unrealized benefit of decreased incidence of cardiac death following AMI.

It is now understood that the evolutionary persistence of allergen caused disease, despite its physiological costs, implies the existence of an adaptive benefit that outweighs the costs. IgE's primary function of combating helminthiasis is the most likely explanation in light of the fact that 70% of the contemporary population resides in the developing world and that helminthiasis remains one of the most common types of disease in that environment, infecting one-third of the total population. Certainly the approximately 200 million people suffering from Schistomiasis benefit from the immune systems' highly evolved strategy against the worm, as did our many predecessors. Indeed, allergy may be the price we must pay for the elaborate protection we have against helminthic parasites. This assumption is based on epidemiological and genetic data that suggest that protection against helminths pre-dates, and in some cases, may even preclude, allergic disease (Barnes et al., 1999).

Isolation of Antigenic Material

In various embodiments of the current invention, antigenic substances will be extracted according to known methods of isolation described in the prior art. The following Example illustrates one method by which antigenic material may be isolated (U.S. Pat. No. 4,396,600). One skilled in the art will appreciate that other techniques may also be used.

The adult worms from which the antigenic proteins will be extracted can be maintained in sufficient quantities for laboratory use in Swiss mice, which can be infested with cercaria from snails like Biomphalaria glabrata. Worms can also be cultured in various other species including hamsters, guinea pigs, monkeys, cows, and the like. Six to eight weeks after infecting the mice, one will then sacrifice the host and perfuse the infected organs with a saline solution, preferably isotonic saline of 0.15 M NaCl. A buffer, preferably sodium phosphate, will then be added to the perfusion fluid (PBS) to maintain the pH of the solution at approximately 6.8. The perfused worms will then be collected and rinsed, preferably with PBS. It is imperative that the worms be rinsed briefly. The amount of rinsing solution and time of contact should be kept to a minimum, sufficient to wash away any blood or tissue, but not so much as to wash away significant worm by-products or potential antigenic materials. The preferred total amount of rinsing solution should be in the range of 10-20 ml per 100 worms. It is also preferred that one-half of the rinsing solution be used for two separate rinsing procedures. Rinsing will be accomplished by placing the collected worms in an appropriately sized sieve, like a wire net, and slowly pouring or spraying the rinsing solution over the worms. This will ensure minimum contact time (preferably on the order of a few seconds) and prevent any significant loss of antigenic material.

The washed worms will then be stored in a saline extraction solution for a period of time sufficient to extract the antigenic material. The previously mentioned pH 6.8 sodium phosphate buffered 0.15 M NaCl (PBS) solution is the preferred extractant. The worms will be placed into the solution of PBS, 5 ml-10 ml of solution per gram of worm, and extracted for 30 minutes to two hours at room temperature. Following the extraction, the solution will be refrigerated. The extraction can also proceed at lower temperatures and therefore can be induced while refrigerated. Refrigeration prevents degradation of the antigenic material for several weeks or months. Furthermore, after extraction, the solution can be frozen, preserving the antigenic material indefinitely.

Additional antigenic material will be obtained from the worms themselves following their removal from the extraction solvent. To accomplish this goal, the worms will be suspended in additional PBS and homogenized. Any conventional tissue grinder homogenizer can be used to macerate the worms, so long as excessive shearing is avoided, since shearing might degrade the high molecular weight (˜50,000) antigenic material. The suspension of homogenized worms and PBS will then be centrifuged at 10,000 cpm to produce a solution containing worm solid and supernatant fluid. The supernatant will then be collected and combined directly with the live worm-derived saline extraction solution.

The antigenic material containing extract or saline extract will then be further purified by passing the extract through a separating means capable of separating substances having molecular weights of 50,000 or more from those having molecular weights below 50,000. The molecular weight of the antigen retrieved from the particular genus species of worm will have to be measured to determine which part of the filtered solution should be saved (that containing the antigenic material) and which will be discarded. The separation means can be an exclusion pore filter, which is capable of retaining solution components having a molecular weight of 50,000 or more; however, the preferred pore size will retain molecules with a molecular weight of 100,000 or more.

One of ordinary skill in the art will understand that it may be necessary to subject the separated saline extract to further purification to obtain antigenic material in sufficiently isolated form to be used as the template for mass protein production via gene cloning. The saline extract will be further purified by means of partition separation, such as gel-filtration or affinity chromatography columns, which are capable of separating high molecular weight molecules from materials with a low molecular weight (gel chromatography) or separation based on the ability to bind particular chemical groups (affinity chromatography). Proteins are most often fractionated by column chromatography, in which a mixture of proteins in solution will be passed through a column containing a porous solid matrix. The different proteins are retarded to different extents by their interaction with the matrix, and they will be collected separately as they flow out of the bottom of the column. Specific antibodies (IgE) will be coupled to a matrix in order to purify protein molecules recognized by the antibodies. Because of the great specificity of all such affinity columns, 1,000-fold to 10,000-fold purifications can sometimes be achieved in a single pass. The resolution of conventional column chromatography is limited by inhomogeneities in the matrices, like cellulose, which causes an uneven flow of solvent through the column. Newer chromatography resins, usually silica based, have been developed in the form of tiny spheres (3-10 um in diameter) that can be packed with a special apparatus to form a uniform column bed. A high degree of resolution is attainable on such high-performance liquid chromatography (HPLC) columns. In HPLC, the solutes equilibrate very rapidly with the interior of the tiny spheres, so solutes with different affinities for the matrix are efficiently separated from one another, even at fast flow rates. This allows most fractionations to be carried out in minutes versus hours (Alberts et al., 1994).

When considering molecular sieve columns, the higher molecular weight molecules are the first fractions that will be eluted form the column. The phosphate buffered saline solution (PBS) will be used to equilibrate the gel or affinity chromatography column and will also be used as the eluant. Depending on the exclusion limit of the particular molecular sieve, the saline extracted antigenic material will either be retained on the column or may be eluted with the voids volume.

Generally, recovery of antigenic proteins in the saline extract ranges from about 10%-30% of the weight of the worms after rinsing. After further purification by gel chromatography, the antigenic protein content ranges from about 2%-4% of the total worm weight. The purified antigenic material will then be used as the active ingredient in a pharmaceutical composition or vaccine for immunizing mammals, especially humans, or utilized to mass-produce proteins via gene cloning.

One of the most important contributions of DNA cloning and genetic engineering is that it is now possible to produce and amplify cellular proteins. Cloning can be performed according to any number of standard protocols well-known in the art. One of the principals underlying cloning is to separate the gene or genes encoding the desired protein and then to clone it. Use of mRNA is one typical starting point. The correct mRNA molecules will be identified by making an appropriate probe, a short DNA molecule corresponding to the specific helminthic antigen/protein. The probe, being complementary to its corresponding mRNA, will hybridize (form hydrogen-bonded base pairs) with it, thereby identifying it. There is a compelling reason for isolating mRNA rather than isolating the gene directly. Genes from eucaryotes contain introns, stretches of DNA that do not code for the protein, because they are cut out of mRNA as it matures. Because prokaryotes lack the enzymes to eliminate introns, they will synthesize an incorrect protein from a eucaryotic gene carrying introns. To determine the sequence of bases needed to make the probe, one will have to determine the sequence of bases needed to make the probe, by determining the sequence of amino acids in the protein. Then the correct sequence of bases for the probe can and will be determined by referring to the genetic code. Because of the redundancy of the code, all of the DNA sequences that designate the desired amino acid sequence must be synthesized and the mixture will be used as a probe (Ingraham and Ingraham, 1996).

Once the correct mRNA has been isolated, reverse transcriptase, the enzyme from retroviruses that uses RNA as a template, will be used to make DNA. This DNA product is termed complimentary DNA or cDNA to indicate that it is a copy of mRNA, not the DNA in the gene itself. The cDNA will be cut with restriction endonucleases and ligated into a bacterial plasmid, which in turn will be inserted into the bacterial host by transformation. In the host, the recombinant DNA molecule will be replicated; and its genes, including the gene encoding for the antigenic protein, will be expressed.

After the antigenic protein-producing bacterial strain is obtained, the job of producing the antigenic protein commercially, has just begun. It will be necessary to modify the plasmid and develop the proper cultural conditions so the bacterial strain carrying the antigen-encoding gene will produce useful amounts of the antigenic protein. Producing huge amounts of foreign proteins is detrimental to the cell, so methods have been devised to trigger E. coli to make antigenic proteins only after a dense culture develops. That is, the growth phase is separated from the production phase. One of the most recent discoveries relates a method for enhancing the production of biologically active proteins and peptides in bacterial cells. The host bacterial cell, containing a plasmid with one or more targeted genes encoding the protein of interest, will be injected with bacteriophage lambda containing one or more copies of the targeted gene(s). The phage increases synthesis of the targeted protein, induces lytic growth of the cell without lysis (until a desired level of protein production is reached), and induces lysis of the producer cell when the desired production level is attained. Super-production will be achieved by cultivating the producer strain cells under culture conditions that delay lytic development of the phage. The biologically active proteins and peptides will subsequently accumulate in a soluble form in the culture medium as the cells of the producer strain are lysed by the phage. An important advantage of infecting producer cells with a bacteriophage is that the phage causes a profound rearrangement of all macromolecular synthesis in the bacterial host cells. By turning off transcription of bacterial genes, phages may increase the copying of the targeted gene, and consequently, increase the output of the desired product (U.S. patent application No. 20,010,044,133).

Because the desired protein made from an expression vector will be produced inside a cell, it must be purified away from the host cell proteins by chromatography following cell lysis; but because it is such a plentiful species in the cell lysate (often 1% to 10% of the total cell protein), the purification is usually easy to accomplish in only a few steps. Utilizing the above-prescribed gene cloning procedure, cells will be induced to make vast quantities of medically useful proteins, such as those utilized in vaccines. Methods have been devised, not only to purify antigenic proteins from a culture, but also to unfold the protein, refold it properly, and properly reglycosylate the resultant protein (Alberts et al., 1994; Ingraham and Ingraham, 1996).

The biological activity of many glycoproteins is highly dependent upon the presence or absence of particular oligosaccharide structures attached attached to the glycoprotein. Furthermore, the glycosylation pattern of a therapeutic glycoprotein can affect numerous aspects of the therapeutic efficacy such as immunogenicity, half-life, bioactivity, and stability. An invention by Robert Bayer provides methods for producing glycopeptides that have a fucosylation pattern, which is substantially identical to the fucosylation pattern of a known glycopeptide. Interestingly, alpha (α) (1,3)-linked fucose has recently been identified as an IgE-binding structure in helminths (Yazdanbakhsh et al., 2002). The method for producing glycopeptides that have a fucosylation pattern will include contacting a glycopeptide having an acceptor for fucosyltransferase with a fucose donor and the fucosyltransferase. The transfer of the fucose onto the glycopeptide will be terminated upon reaching a desired level of fucosylation. Among the uses of this aspect of the invention is the duplication of therapeutically relevant glycopeptide structures. This will allow switching from a production cell line with adequate glycosylation capabilities, but limited in expression level, to a production cell line that has the capability of producing significantly greater amounts of product, but yielding an inferior glycosylation pattern. The glycosylation pattern will be modified in vitro to match that of the desired product. The yield of desired glycosylated product may then be increased substantially for a given bioreactor size, impacting both production economics and plant capacity. The particular glycopeptide used in the methods of the invention is generally not a critical aspect of the invention. The glycopeptide may be a fragment or a full-length glycopepetide. Typically, the glycopeptide is one that has therapeutic use (U.S. patent application No. 20,020,019,342).

Presently, there are numerous biotechnology companies who specialize in mass-producing immunologically based protein antigens on a commercial basis. Many international protein design & manufacturing companies provide protein expression services for the production, purification, and optimization of recombinant and native proteins from mg to kg quantities. The scope of available services usually includes gene cloning, strain construction, expression optimization, development of purification schemes, and production of the product. These services will be utilized if one does not have the equipment or the capability to isolate a desired protein (antigenic proteins in this case), purify it utilizing column chromatography, designing an appropriate probe to identify the mRNA that codes for the protein, inserting the desired mRNA into a host cell, and mass producing the protein utilizing genetic engineering procedures. Furthermore, these companies can purify the extracted mass-produced protein (possibly utilizing affinity chromatography), identify the structure of the native glycosylated protein, and synthesize the exact fucosylated glycoprotein for utilization in an allergy treatment or vaccine.

A pharmaceutical composition or vaccine for the treatment and prevention of allergies and asthma will primarily be comprised of equal amounts of antigenic proteins (derived using the aforementioned procedures) from 3-5 different helminth sources. The helminthes of choice will include the aforementioned worms that are rare and induce a strong immunogenic response in human immune systems (i.e. Dicrocoelium dentriticum, various genus-species of Schistosomes, etc.). The carrier or medium in which the shot/vaccine will be prepared can be any suitable sterile medium, like sterile phosphate-buffered saline or PBS. The composition will also be fortified with a suitable adjuvant, such as BCG (Mycobacterium bovis, strain Bacille Calmette-Guerin) or other adjuvants that induce cell-mediated immunity and increased immunogenic response in the host. BCG is the preferred adjuvant, because it was determined that BCG acted as a potent adjuvant in association with antigenic material (U.S. Pat. No. 4,656,033).

The components of the pharmaceutical composition or vaccine (the purified glycosylated antigenic protein, the PBS medium, and the adjuvant BCG that increases immunogenic response in the host) will be combined to produce a solution containing 1 mg of antigen and 3.5 times 10 to the 7'th (3.5×10 Exp.7) of colony forming units (CFU) of BCG, both of which will be suspended in 0.1 ml of PBS. Suitable doses of the antigenic material range from about 1 ug to 20 mg per kilogram of the recipients body weight, preferably between 10 ug and 30 ug. Immunologically effective amounts of the pharmaceutical composition will be injected subcutaneously and/or intradermally into the recipient's buttocks or upper arm at his or her first visit. A follow-up appointment will be set for 3-4 weeks later, when blood will be drawn to measure total serum IgE by utilizing the ELISA test. Once blood is drawn for the ELISA, then the patient will receive an additional inoculation of the same dose used for the initial shot. Another follow-up appointment will be set for 3-4 weeks after the booster shot, at which time blood will be drawn again to measure total serum IgE concentration using the ELISA test. Another follow up appointment will be set for one week later to examine and compare the total serum IgE, measured before and after the second inoculation. The change in measured IgE levels will be compared to the dose of the pharmaceutical composition injected and contrasted with the desired level of total serum IgE. Since it is preferable to maintain an IgE level between 3,000 IU and 15,000 IU, the patient will be injected with a sufficient amount of the pharmaceutical compound to raise total serum IgE to a level of 15,000 IU; thereby assuring that the preferable range of total serum IgE is maintained above the recommended level of 3,000 IU, for a period of 3-6 months. The appropriate dose necessary to achieve a total serum IgE level of 15,000 IU will be illustrated in the following two examples.

The following Examples illustrate various embodiments of the present invention and are not intended in any way to limit the invention.

### EXAMPLE 1

The patient to be inoculated is a man weighing 100 kg (220 lbs.) Utilizing the mean (20 ug) of the preferred range of 10 ug-30 ug per kg of body weight as the optimal amount of antigenic material required in the pharmaceutical composition, the inoculate will contain 2,000 ug or, equivalently, 2 mg of the antigenic substance, in order to produce the desired immune response. Using the before mentioned concentration of the preferred inoculate (1 mg of antigen combined with 3.5×10 Exp.7 CFU of BCG, suspended in 0.1 ml of PBS), the volume of the pharmaceutical composition necessary to provide the desired immunological response for a man weighing 100 kg is approximately 0.2 ml. Therefore, the initial and second inoculation will involve the transdermal injection of 0.2 ml of the composition. If, for example, the ELISA test performed before the second inoculation results in a total serum IgE level of 5,000 IU and the level is measured to be 10,000 IU following the second shot, then the third inoculation will also be approximately 0.2 ml, since the total IgE level was raised 5,000 IU following the second inoculation and the same rise in serum IgE will be desired with the third treatment. Since the desired level of serum IgE is approximately 15,000 IU, then an increase of 5,000 IU is desired. Since the total serum IgE was raised 5,000 IU after the 2'nd shot and one will desire the same increase from the 3'rd inoculation, then the same dose will be required as that given in the second shot or specifically 0.2 ml of the pharmaceutical composition.

### EXAMPLE 2

One is presented with a toddler weighing 5 kg. Again, one will use 20 ug/kg as the preferred amount necessary to invoke the desired immune response and the previously mentioned antigen, BCG, and PBS concentrations for the pharmaceutical composition will remain unchanged. The amount of antigenic protein necessary to provide the desired result will then be calculated and found to be 100 ug or 0.1 mg with a resulting injection volume of approximately 0.01 ml necessary to produce the desired effect. The same procedures will be followed for the toddler as described in the treatment regime for the 100 kg man. An ELISA test will be performed prior to and after the 2'nd inoculation to measure total serum IgE. The change in serum IgE concentration will be noted and an appropriate dose for the third inoculation will be calculated as outlined in example 1 for the adult. The toddler will then receive a third inoculation designed to elevate total IgE to a level of approximately 15,000 IU. The patient will be instructed to return in approximately 3-6 months for another ELISA test that will ascertain the best dose to be administered every 3-6 months in order to maintain total serum IgE levels of approximately 3,000 IU-15,000 IU.

While a number of preferred embodiments of the invention and variations thereof have been described in detail, other modifications and methods of use will be readily apparent to those of skill in the art. Accordingly, it should be understood that various applications, modifications, and substitutions may be made of equivalents without departing from the spirit of the invention or the scope of the claims.

What is claimed is:

1. A pharmaceutical formulation for preventing or treating allergies or asthma in a mammal comprising at least one helminth-based agent, wherein said helminth-based agent is capable of ameliorating the allergic reaction to a plurality of antigens.

2. The formulation of claim 1, further comprising at least one pharmaceutically acceptable compound selected from the group consisting of adjuvants, carriers and diluents.

3. The formulation of claim 1, wherein said helminth-based agent is an immunogenic amount of a helminthic antigen.

4. The formulation of claim 3, wherein said helminthic antigen is an isolated protein selected from the group consisting of nematodes, trematodes and cestodes.

5. The formulation of claim 3, wherein said helminthic antigen is a protein isolated from a helminth, wherein said helminth is parasitic to humans.

6. The method of claim 3, wherein said helminthic antigen is an isolated protein selected from the group consisting of Capillaria hepatica and Dicrocoelium dendtriticum.

7. The formulation of claim 1, wherein said helminth-based agent is an effective amount of a nucleic acid molecule encoding at least one epitope of a helminthic organism.

8. The formulation of claim 1, wherein said helminth-based agent comprises a protein isolated from a helminth, wherein said protein is a recombinant cell transformed with a nucleic acid molecule encoding said protein.

9. The formulation of claim 1, wherein said helminth-based agent is an antibody directed to at least one epitope of a helminthic antigen.

10. The formulation of claim 9, wherein said antibody is a monoclonal antibody.

11. The formulation of claim 1, wherein said pharmaceutical formulation is in a form selected from the group consisting of injectable fluids, suppositories, powder, tablets, capsules, syrups, suspensions, liquids and elixirs.

12. A vaccine for preventing allergies or asthma in a mammal comprising the pharmaceutical formulation of claim 1 in an amount sufficient to regulate IgE.

13. A method of preventing or treating allergies or asthma in a mammal comprising administering a therapeutically effective dose of the pharmaceutical formulation of claim 1 to said mammal.

14. The method of claim 13, wherein said pharmaceutical formulation is administered by a route which results in systemic absorption of an immunogenic amount of said pharmaceutical formulation.

15. The method of claim 13, wherein said pharmaceutical formulation is administered intradermally, intravenously, orally or rectally.

16. A method of immunizing a human against IgE-regulated allergic reactions by administering an effective dose of the pharmaceutical formulation of claim 1 to said human.

17. The method of claim 13, wherein said human is less than one year old.

18. The method of claim 13, wherein said administering occurs within two weeks after birth.

19. A method of relieving the symptoms of allergy in a mammal comprising administering a therapeutically effective dose of the pharmaceutical formulation of claim 1 to said mammal when experiencing said symptoms

20. A method of competitively inhibiting allergen-specific IgE in a mammal comprising administering a therapeutically effective dose of the pharmaceutical formulation of claim 1 to said mammal.

21. The method of claim 16, further comprising measuring total serum IgE levels and serum levels of IgE specific to allergens.

22. The method of claim 21, wherein said measuring is performed by ELISA or enzyme-linked immunosorbent assay testing.

23. The method of claim 16, wherein said therapeutically effective dose of the pharmaceutical formulation is determined by measuring said mammal's IgE levels and administering a dose sufficient to provide a desired level, wherein said desired level is greater than about 1500 IU/ml.

24. The method of claim 23, wherein said desired level is about 3000 IU/ml.
